SEQUENCE LISTING (16 sequences)

SEQ ID NO: 1 - protein, 846 aa, Kluyvera sp.
MKNRNRMIVNGIVTSLICCSSLSALAASPPTEVKIVRDEYGMPHIYADDTYRLFYGYGYVVAQDRLFQMEMARRSTQGTVSEVLGKAFVSFDKDIRQNYWPDSIRAQIASLSAEDKSILQGYADGMNAWIDKVNASPDKLLPQQFSTFGFKPKHWEPFDVAMIFVGTMANRFSDSTSEIDNLALLTALKDKYGKQQGMAVFNQLKWLVNPSAPTTIAARESAYPLKFDLQNTQTAALLPRYDQPAPMLDRPAKGTDGALLALTADQNRETIAAQFAQSGANGLAGYPTTSNMWVIGKNKAQDAKAIMVNGPQFGWYAPAYTYGIGLHGAGYDVTGNTPFAYPGLVFGHNGTISWGSTAGFGDDVDIFAEKLSAEKPGYYQHNGEWVKMLSRKETIAVKDGQPETFTVWRTLHGNVIKTDTATQTAYAKARAWDGKEVASLLAWTHQMKAKNWPEWTQQAAKQALTINWYYADVNGNIGYVHTGAYPDRQPGHDPRLPVPGTGKWDWKGLLSFDLNPKVYNPQSGYIANWNNSPQKDYPASDLFAFLWGGADRVTEIDTILDKQPRFTADQAWDVIRQTSRRDLNLRLFLPALKDATANLAENDPRRQLVDKLASWDGENLVNDDGKTYQQPGSAILNAWLTSMLKRTVVAAVPAPFGKWYSASGYETTQDGPTGSLNISVGAKILYEALQGDKSPIPQAVDLFGGKPQQEVILAALDDAWQTLSKRYGNDVTGWKTPAMALTFRANNFFGVPQAAAKEARHQAEYQNRGTENDMIVFSPTSGNRPVLAWDVVAPGQSGFIAPDGKADKHYDDQLIMYESFGRKSLWLTPQDVDEHKESQEVLQVQR

SEQ ID NO: 2 - DNA, 2541 bp, Kluyvera sp.
  source 1..2541: mol_type = genomic DNA
atgaaaaatagaaatcgtatgatcgtgaacggtattgtgacttccctgatctgttgttctagcctgtcagcgctggcggcaagcccgccaaccgaggttaagatcgttcgcgatgaatacggcatgccgcatatttacgccgatgatacctatcgactgttttacggctatggctacgtggtggcgcaggatcgcctgttccagatggaaatggcgcgccgcagtactcaggggaccgtctccgaggtgctgggcaaagcattcgtcagttttgataaagatattcgccagaactactggccggattctattcgcgcgcagatagcttccctctccgctgaggataaatccattctgcagggctatgccgatggcatgaatgcgtggatcgataaagtgaacgccagccccgataagctgttaccccagcagttctccacctttggttttaaacccaagcattgggaaccgtttgatgtggcgatgatttttgtcggcaccatggcgaaccgtttctctgacagcaccagcgaaattgataacctggcgctgctgacggcgctaaaagataaatacggcaagcagcagggcatggcggtctttaaccagctgaaatggctggttaatccttccgcgccaaccaccattgcggcgcgggaaagcgcctatccgctgaagtttgatctgcaaaacacgcaaacggcggcgctgctgccgcgctacgaccagccggcaccgatgctcgaccgcccggcaaaagggaccgatggcgcgctgctggcgctgaccgccgatcagaaccgggaaactatcgccgcgcagttcgcgcaaagcggcgctaacggcctggctggctacccgaccactagcaatatgtgggtgattggcaaaaacaaagcccaggatgcgaaggccattatggtcaatgggccgcagtttggttggtatgcgccggcgtacacctacggtatcggcctgcacggcgcgggctatgacgtcaccggcaatacgccgtttgcctatccgggcctcgtttttggtcacaacggcaccatttcatggggatccaccgccggttttggtgatgatgtcgatatctttgccgaaaaactttccgccgagaagccgggctattaccagcataacggcgagtgggtgaagatgttgagccgcaaggagactattgcggtcaaagacggccagccggagacctttaccgtttggcgcacgctgcacggcaacgtcattaaaaccgatactgcgacgcagaccgcctatgccaaagcgcgcgcctgggatggcaaagaggtggcgtccctgctggcgtggacgcaccagatgaaggccaaaaactggccggagtggacgcagcaggcggccaaacaggcgctgaccattaactggtactacgccgatgtgaacggcaatatcggctatgtgcataccggcgcctatccggatcgccagcccggccacgacccgcgtttgccggttcccggcactggaaaatgggactggaaagggttgctgtcgtttgatttgaatccgaaagtgtataacccgcagtcgggctatatcgccaactggaacaactcgccgcaaaaagactacccggcctctgatctgttcgcgttcctgtggggcggtgcggatcgagttactgagatcgacacgatcctcgataagcaaccgcgcttcaccgccgatcaggcgtgggatgtgatccgccaaaccagccgtcgggatctcaacctgcggttgttcttaccggcgctgaaggacgccaccgcgaacctggcggaaaacgatccgcgccgccaactggtggataaactggcgagctgggacggtgaaaaccttgtcaacgatgacggaaaaacctatcagcaaccgggatcggcgattcttaacgcctggctgaccagcatgctcaagcgcacggtggttgccgcggtcccagcgccgtttggcaagtggtacagcgccagtggctatgaaaccacccaggacgggccaaccggctcgctgaacatcagcgtgggggcgaaaatcctctacgaagctctgcagggtgataagtcgccaatcccgcaggcggtcgatctgtttggcgggaaaccgcagcaggaagtgatactggcggcgctggacgacgcttggcagacgctgtcaaaacgctacggtaacgacgtcaccggctggaaaacccctgccatggcgcttaccttccgggccaataacttcttcggcgtgccgcaggcggcagcaaaagaggcgcgtcatcaggcggagtaccagaaccgcggtacggaaaacgacatgattgtcttctcaccgacgtcgggtaaccgcccggttcttgcctgggatgtggtggcgccggggcaaagcggttttatcgcgccggatggcaaagccgataagcactatgacgatcagctgataatgtacgagagctttggccgtaaatcgctgtggttaacgcctcaggacgttgacgagcacaaagagtctcaggaagtgctgcaggtacagcgctaa

SEQ ID NO: 3 - DNA, 28 bp, synthetic construct
  source 1..28: mol_type = other DNA
cggaattcatgaaaaaccgcaatcgcat

SEQ ID NO: 4 - DNA, 27 bp, synthetic construct
  source 1..27: mol_type = other DNA
ccaagcttttagcgctgcacctgcagc

SEQ ID NO: 5 - DNA, 27 bp, synthetic construct
  source 1..27: mol_type = other DNA
ggcgaaccgtaaatctgacagcaccag

SEQ ID NO: 6 - DNA, 25 bp, synthetic construct
  source 1..25: mol_type = other DNA
atggtgccgacaaaaatcatcgcca

SEQ ID NO: 7 - DNA, 25 bp, synthetic construct
  source 1..25: mol_type = other DNA
tgggccgcagcgcggttggtatgcg

SEQ ID NO: 8 - DNA, 25 bp, synthetic construct
  source 1..25: mol_type = other DNA
ttgaccataatggccttcgcatcct

SEQ ID NO: 9 - DNA, 23 bp, synthetic construct
  source 1..23: mol_type = other DNA
caccgccggttatggtgatgatg

SEQ ID NO: 10 - DNA, 25 bp, synthetic construct
  source 1..25: mol_type = other DNA
gatccccatgaaatggtgccgttgt

SEQ ID NO: 11 - DNA, 23 bp, synthetic construct
  source 1..23: mol_type = other DNA
cgccaactggaaaaactcgccgc

SEQ ID NO: 12 - DNA, 26 bp, synthetic construct
  source 1..26: mol_type = other DNA
atatagcccgactgcgggttatacac

SEQ ID NO: 13 - DNA, 31 bp, synthetic construct
  source 1..31: mol_type = other DNA
cgggccaacctattcgctgaacatcagcgtg

SEQ ID NO: 14 - DNA, 25 bp, synthetic construct
  source 1..25: mol_type = other DNA
tcctgggtggtttcatagccactgg

SEQ ID NO: 15 - DNA, 25 bp, synthetic construct
  source 1..25: mol_type = other DNA
cgggccaacccgctcgctgaacatc

SEQ ID NO: 16 - DNA, 25 bp, synthetic construct
  source 1..25: mol_type = other DNA
tcctgggtggtttcatagccactgg

(19) United States
(12) Patent Application Publication
(10) Pub. No.: US 2026/0117216 A1
(43) Pub. Date: Apr. 30, 2026

(54) MUTANT PENICILLIN ACYLASE AND USES THEREOF

(71) Applicant: XINGZHI COLLEGE ZHEJIANG NORMAL UNIVERSITY, Lanxi (CN)

(72) Inventors: Zhonghua YANG, Lanxi (CN); Xiaoyan YIN, Lanxi (CN)

(73) Assignee: XINGZHI COLLEGE ZHEJIANG NORMAL UNIVERSITY, Lanxi (CN)

(21) Appl. No.: 19/344,553

(22) Filed: Sep. 30, 2025

(30) Foreign Application Priority Data
Oct. 8, 2024 (CN) 2024113960690

(51) Int. Cl. C12N 9/84; C12N 15/70; C12P 35/04; C12P 37/00; C12R 1/19

(52) U.S. Cl. CPC C12N 9/84; C12N 15/70; C12P 35/04; C12P 37/00; C12R 2001/19; C12Y 305/01011

(57) ABSTRACT

A mutant penicillin acylase and uses thereof are provided. Compared to the amino acid sequence set forth in SEQ ID NO: 1, the mutant includes at least one mutation selected from the group consisting of: F146αK, F24βR, F71βY, N241βK, G385βY, and G385βR. By introducing mutations into the penicillin acylase derived from Kluyvera citrophila, the invention obtains a mutant enzyme exhibiting enhanced and well-coordinated hydrolytic and synthetic activities. It can be used for the synthesis of β-lactam antibiotics, particularly for the one-step preparation of amoxicillin from penicillin potassium salt, thereby avoiding the isolation of the intermediate 6-APA. The present invention provides a key enzyme for the efficient production of β-lactam antibiotics and is poised to significantly advance the innovation of their manufacturing technology.

### CROSS REFERENCE TO THE RELATED APPLICATIONS

This application is based upon and claims priority to Chinese Patent Application No. 202411396069.0, filed on Oct. 8, 2024, the entire of which are incorporated herein by reference.

### SEQUENCE LISTING

The instant application contains a Sequence Listing which has been submitted in XML format via EFS-Web and is hereby incorporated by reference in its entirety. Said XML copy is named FSJZ0103S_Sequence_Listing, created on Sep. 28, 2025, and is 18,348 bytes in size.

### TECHNICAL FIELD

The present invention pertains to the field of enzyme catalysis technology, and more particularly, to a mutant penicillin acylase and uses thereof.

### BACKGROUND

Beta-lactam antibiotics (β-lactams) refer to a major class of antibiotics characterized by a beta-lactam ring in their chemical structure. These antibiotics offer advantages such as potent bactericidal activity, low toxicity, a broad range of indications, and excellent clinical efficacy. However, naturally occurring beta-lactam antibiotics, such as penicillins and cephalosporins, suffer from drawbacks including a narrow antibacterial spectrum, acid intolerance, a high propensity for inducing drug resistance, and a tendency to cause allergic reactions. Semi-synthetic beta-lactam antibiotics, like semi-synthetic penicillins and semi-synthetic cephalosporins, have overcome these limitations and have become the primary class of antibiotics used in current clinical practice.

The preparation of semi-synthetic β-lactam antibiotics using enzyme catalysis technology enables a more efficient, green, and sustainable production process, yielding products of superior quality compared to traditional chemical synthesis. Penicillin acylase is the key enzyme catalyzing the synthesis of such products. The enzymatic synthesis of semi-synthetic penicillins, such as ampicillin and amoxicillin, currently primarily involves a two-step process. First, penicillin G/V or its salts are hydrolyzed by a hydrolysis-specific penicillin acylase to produce 6-APA (6-aminopenicillanic acid). Subsequently, 6-APA reacts with an acyl donor side chain (e.g., D-p-hydroxyphenylglycine methyl ester) catalyzed by a synthesis-specific penicillin acylase to form the semi-synthetic β-lactam antibiotic. For semi-synthetic cephalosporins, the synthesis mainly involves the reaction of cephalosporin intermediate cores, such as 7-ACA (7-aminocephalosporanic acid) and 7-ADCA (7-aminodeacetoxycephalosporanic acid), with an acyl donor, catalyzed by penicillin acylase. Penicillin Acylase (PA, EC 3.5.1.11) is the key enzyme in this preparation technology. Currently, the existing art employs two distinct types of penicillin acylase—a hydrolysis-specific enzyme and a synthesis-specific enzyme—to catalyze the two steps separately in a sequential manner: the hydrolysis step for producing 6-APA (or 7-ACA, 7-ADCA) and the subsequent condensation step between the core intermediate and the side chain. This two-step process is not only prolonged but also requires the separation of intermediates, resulting in a relatively complex production workflow.

### SUMMARY

In view of the problems associated with the prior art, the present invention provides a mutant penicillin acylase and uses thereof, aiming to resolve the issues existing in the current techniques. The mutant provided herein is capable of catalyzing the reaction between a β-lactam starting material (such as penicillin or cephalosporin) and an activated acyl donor to synthesize semi-synthetic β-lactam antibiotics, including semi-synthetic penicillins and semi-synthetic cephalosporins. This enables a novel technology for the rapid and highly efficient synthesis of semi-synthetic β-lactam antibiotics.

The invention is achieved as follows. Provided is a mutant penicillin acylase which, compared to the amino acid sequence set forth in SEQ ID NO: 1, comprises at least one mutation selected from the group consisting of: F146αK, F24βR, F71βY, N241βK, G385βY, and G385βR. Amino acid single-letter code designations: F: Phenylalanine; K: Lysine; R: Arginine; Y: Tyrosine; N: Asparagine; G: Glycine.

The present invention further provides a nucleotide sequence encoding any of the aforementioned mutant penicillin acylases.

The present invention also provides the use of the aforementioned mutant penicillin acylase in the preparation of β-lactam antibiotics.

Preferably, the β-lactam antibiotics include semi-synthetic penicillins and semi-synthetic cephalosporins. The semi-synthetic penicillins include amoxicillin, ampicillin, or pivampicillin. The semi-synthetic cephalosporins include cefalexin, cefprozil, cefaclor, cefradine, cefadroxil, cefamandole, cefazolin, cefonicid, cephalothin, or cephaloglycin.

Preferably, any one of the aforementioned mutant penicillin acylases is used as the sole penicillin acylase in the reaction system to catalyze the reaction between a penicillin potassium salt and an acyl donor, thereby synthesizing semi-synthetic penicillin β-lactam antibiotics in a single step.

Preferably, the acyl donor includes phenylglycine methyl ester or p-hydroxyphenylglycine methyl ester.

Preferably, any one of the aforementioned mutant penicillin acylases is used as the penicillin acylase to catalyze the reaction of 7-ACCA or 7-ADCA with phenylglycine methyl ester, thereby synthesizing semi-synthetic cephalosporin β-lactam antibiotics.

The present invention involves introducing mutations into amino acid residues within the substrate-binding region of the wild-type penicillin acylase. These modifications enhance substrate binding affinity and reduce steric hindrance effects at the substrate-binding site. Concurrently, the mutations lower the transition-state free energy between the substrate and the active site in the catalytic center, thereby facilitating the conversion of the reaction intermediate to the final product. This dual effect promotes the accumulation of the end product and ultimately significantly enhances the capability of the mutant penicillin acylase to synthesize semi-synthetic β-lactam antibiotics.

In summary, the advantages and positive effects of the present invention are as follows: By mutating the penicillin acylase derived from Kluyvera citrophila, the invention has yielded mutant enzymes exhibiting enhanced hydrolytic and synthetic activities, better coordination between these two activities, and improved stability. These mutants can be used for the synthesis of β-lactam antibiotics, including semi-synthetic penicillins (such as amoxicillin, ampicillin, or pivampicillin) and semi-synthetic cephalosporins (such as cefalexin, cefprozil, cefaclor, and cefradine). Notably, they enable a one-step synthesis process for antibiotics like amoxicillin and ampicillin directly from penicillin potassium salt, avoiding the need for isolating intermediates such as 6-APA. The present invention provides a key enzyme for the efficient production of β-lactam antibiotics and is poised to significantly advance innovation in β-lactam antibiotic manufacturing technology.

### BRIEF DESCRIPTION OF THE DRAWINGS

FIG. 1 shows the amino acid sequence (SEQ ID NO: 1) of the wild-type penicillin acylase enzyme.

FIG. 2 is a schematic diagram of the construction of the recombinant plasmid pET28a-kcPA.

FIG. 3 displays the agarose gel electrophoresis analysis of the PCR-amplified recombinant plasmid.

FIG. 4 presents the SDS-PAGE electrophoresis results of protein expression in E. coli BL21(DE3)/pET28a-kcPA cells.

FIG. 5 illustrates the HPLC chromatogram of amoxicillin synthesized in a one-step reaction catalyzed by KcPA from penicillin potassium salt in Embodiment 3.

FIG. 6 shows the changes in substance concentrations during the reaction in Embodiment 4.

FIG. 7 shows the changes in substance concentrations during the reaction in Embodiment 5.

FIG. 8 shows the changes in substance concentrations during the reaction in Embodiment 6.

FIG. 9 shows the changes in substance concentrations during the reaction in Embodiment 7.

### DETAILED DESCRIPTION OF THE EMBODIMENTS

For clarity and to avoid limiting the scope of the invention, all numerical values expressing quantities, percentages, or other parameters in this application shall be interpreted as being modified by the term “about.” Unless explicitly stated otherwise, all numerical parameters in the specification and claims are approximations that may vary depending on the desired properties sought to be achieved. These numerical parameters should be interpreted in light of the reported significant digits and conventional rounding techniques. As used herein, “about” means within ±10% of a stated value or range, preferably within ±5%.

Ambient temperature: Unless otherwise specified, experiments are conducted under ambient conditions (natural room temperature without additional cooling/heating), typically defined as 10 to 30° C., preferably 15 to 25° C. Abbreviations: “min” (minute), “s” (second), “U” (enzyme activity unit), “mM” (millimolar per liter), “M” (molar per liter), “rpm” (revolutions per minute), “mol” (mole), “μg” (microgram), “mg” (milligram), “g” (gram), “μL” (microliter), “mL” (milliliter), “bp” (base pair), “LB medium” (Luria-Bertani medium), “Kan50” (medium containing 50 μg/mL kanamycin).

For experimental methods not specified with specific conditions in the examples, conventional conditions were typically followed, such as those described in the “Molecular Cloning: A Laboratory Manual” (Chinese Edition) (J. Sambrook, M. R. Green, translated by H. Fuchu, 4th edition, Beijing: Science Press, 2017) and in New England Biolabs (NEB) kits.

The present invention discloses a mutant penicillin acylase and its applications. The technical solution provided herein eliminates the need for intermediate separation, significantly simplifies the production process, improves product yield, substantially reduces production costs, and is poised to revolutionize the semi-synthetic antibiotic industry. The following will describe the technical solutions of the present invention clearly and completely in conjunction with the embodiments of the present invention.

### Embodiment 1

Construction of the Prokaryotic Expression for the Mutant of Penicillin Acylase from Kluyvera citrophila, and its Functional Characterization
1. Construction of Wild-Type PA Expression Vector pET28a-kcPA

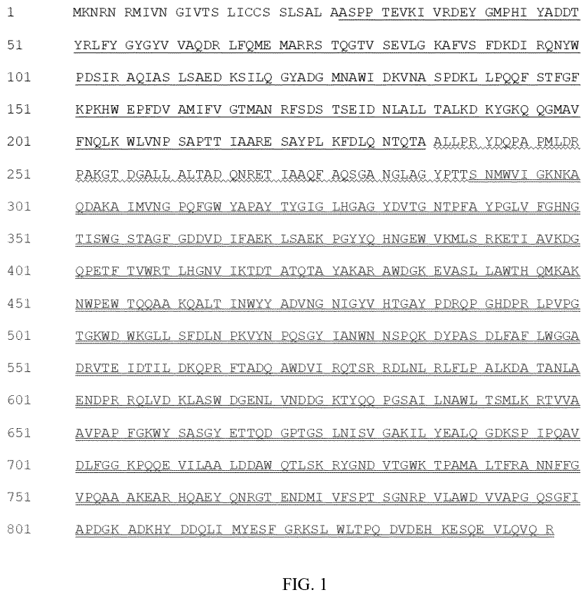

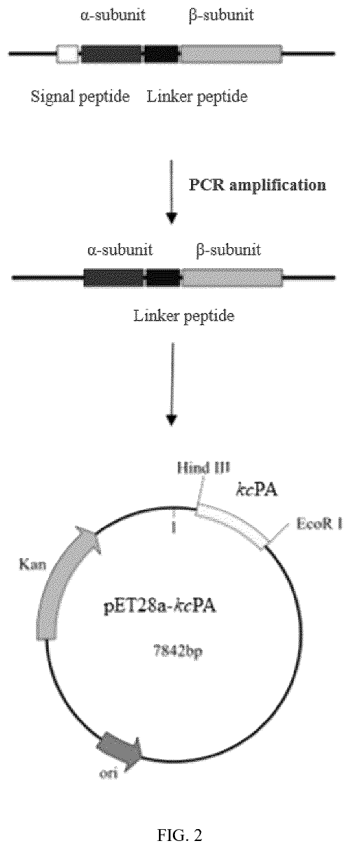

The wild-type penicillin acylase used in this Embodiment originates from Kluyvera citrophila ATCC 21285. Its amino acid sequence (SEQ ID NO: 1) comprises four domains, the sequence from the N-terminus to the C-terminus of the protein is as follows: Signal peptide: Positions 1-26. α-subunit: Positions 27-235 (209 amino acids). Linker peptide: Positions 236-289 (54 amino acids). β-subunit: Positions 290-846 (557 amino acids). Refer to FIG. 1 for domain annotations (single underline: α-subunit; wavy line: linker; double underline: β-subunit). The nucleotide sequence is provided as SEQ ID NO: 2.

The recombinant plasmid pET28a-kcPA was constructed as illustrated in FIG. 1. Using genomic DNA from K. citrophila ATCC 21285 as a template, primers were designed based on the nucleotide sequence of PA (SEQ ID NO: 2): Forward primer (SEQ ID NO: 3): 5′-CGG/AATTCATGAAAAACCGCAATCGCAT-3′. Reverse primer (SEQ ID NO: 4): 5′-CCA/AGCTTTTAGCGCTGCACCTGCAGC-3′. EcoRI and HindIII restriction enzyme sites were introduced into the primer sequences (the underlined bases indicate the restriction enzyme recognition sites), and the target fragment of the wild-type PA was then amplified by PCR.

### PCR Reaction Mixture

| Component | Volume | |
|---|---|---|
| Sterile ddH₂O | 10 | μL |
| template | 0.5 | μL |
| Forward primer (10 μM) | 1 | μL |
| Reverse primer (10 μM) | 1 | μL |
| 2 × Taq Polymerase | 12.5 | μL |
| Total reaction volume | 25 | μL |

PCR Thermal Cycling as following:

| | | |
|---|---|---|
| \textcircled{1}. 95° C. | 5 | min |
| \textcircled{2}. 95° C. | 45 | s |
| \textcircled{3}. 60° C. | 50 | s |
| \textcircled{4}. 72° C. | 90 | s |
| \textcircled{5}. Go to \textcircled{2} | 30 | cycles |
| \textcircled{6}. 72° C. | 10 | min |

| | |
|---|---|
| \textcircled{7}. 4° C. | forever |

The empty plasmid pET28a and PCR-amplified PA DNA fragment were subjected to double restriction enzyme digestion using EcoRI and HindIII restriction enzymes.

### Double Enzyme Digestion System:

| Component | Volume/Amount | |
|---|---|---|
| 10 × Buffer | 5 | μL |
| EcoRI | 1.5 | μL |
| HindIII | 1.5 | μL |
| Empty vector or PCR product | 42 | μL |
| Total | 50 | μL |

The double restriction enzyme digestion was carried out at 37° C. for 1 hour, followed by enzyme inactivation at 80° C. for 20 minutes. The digested products were purified and quantified via agarose gel electrophoresis, yielding approximate concentrations of 50 ng/μL for the pET28a vector and 140 ng/μL for the kcPA insert.

The purified fragments were ligated using T4 DNA ligase in a 16° C. metal bath overnight to generate the recombinant plasmid pET28a-kcPA. The recombinant plasmid was introduced into competent E. coli DH5α cells via heat shock transformation.

Connection reaction system of the target fragment and the linearized vector:

| Component | Volume | |
|---|---|---|
| 10 × Buffer | 1 | μL |
| T4 DNA ligase | 1 | μL |
| Linearized vector | 4 | μL |
| Insert fragment | 4 | μL |
| Total | 10 | μL |

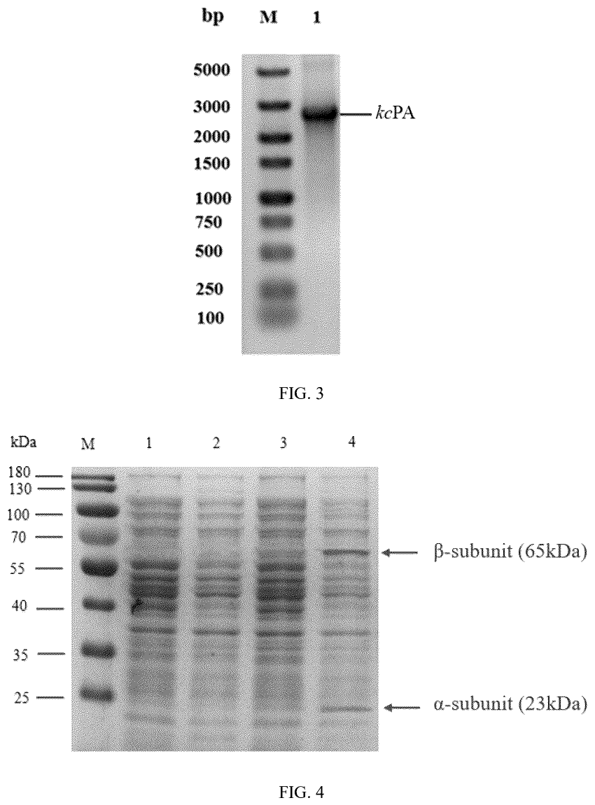

To verify successful plasmid transformation, single colonies were picked from Kan50-LB agar plates and inoculated into Kan50-LB liquid medium. On the following day, plasmids were extracted using a plasmid extraction kit and subjected to PCR verification. Agarose gel electrophoresis confirmed the presence of a 2500-bp target band (see FIG. 3). The validated expression vector pET28a-kcPA was then transformed into E. coli BL21(DE3) to generate the recombinant strain E. coli BL21(DE3)/pET28a-kcPA for wild-type PA expression.

### 2. Generation of Mutant Expression Vectors

In this embodiment, a total of 18 mutants were obtained through site-directed mutagenesis, as shown in the table below. The notation “F146αK” indicates that the amino acid at position 146 on the a subunit was changed from F (Phenylalanine) to K (Lysine). The explanation for other mutation sites follows the same logic. “&” means “and”.

TABLE 1
Mutants and Corresponding Mutation Sites

| Mutant ID | Mutation Sites |
|---|---|
| KcPA | Wild-type |
| KcPA | Single-point mutants: F146αK, F24βR, F71βY, N241βK, |
| 01-06 | G385βY, G385βR |
| KcPA | Double-point mutants: F146αK combined with one of F24βR, |
| 07-11 | F71βY, N241βK, G385βY, or G385βR |
| KcPA 12 | Triple-point mutant: F146αK & F24βR & F71βY |
| KcPA 13 | Triple-point mutant: F146αK & N241βK & G385βY |
| KcPA 14 | Triple-point mutant: F146αK & N241βK & G385βR |
| KcPA 15 | Triple-point mutant: F146αK & F71βY & N241βK |
| KcPA 16 | Quadruple-point mutant: F146αK & F71βY & N241βK & |
| | G385βY |
| KcPA 17 | Quintuple-point mutant: F146αK & F24βR & F71βY & |
| | N241βK & G385βY |
| KcPA 18 | Quintuple-point mutant: F146αK & F24βR & F71βY & |
| | N241βK & G385βR |

First, primers corresponding to each mutation site were designed. Then, Using the wild-type PA target fragment as the initial template, site-directed mutagenesis was performed with the NEB Q5® Site-Directed Mutagenesis Kit (Q5 SDM Kit). The primers for each mutation site are listed below (lowercase letters indicate mutated nucleotides):

F146αK,
Forward (F):
SEQ ID NO: 5
5′-GGCGAACCGTaaaTCTGACAGCACCAG-3′;
Reverse (R):
SEQ ID NO: 6
5′-ATGGTGCCGACAAAAATCATCGCCA-3′;
F24βR,
Forward (F):
SEQ ID NO: 7
5′-TGGGCCGCAGcgcGGTTGGTATGCG-3′;
Reverse (R):
SEQ ID NO: 8
5′-TTGACCATAATGGCCTTCGCATCCT-3′;
F71βY,
Forward (F):
SEQ ID NO: 9
5′-CACCGCCGGTtatGGTGATGATG-3′;
Reverse (R):
SEQ ID NO: 10
5′-GATCCCCATGAAATGGTGCCGTTGT-3′;
N241βK,
Forward (F):
SEQ ID NO: 11
5′-CGCCAACTGGaaaAACTCGCCGC-3′;
Reverse (R):
SEQ ID NO: 12
5′-ATATAGCCCGACTGCGGGTTATACAC-3′;
G385βY,
Forward (F):
SEQ ID NO: 13
5′-CGGGCCAACCtatTCGCTGAACATCAGCGTG-3′;
Reverse (R):
SEQ ID NO: 14
5′-TCCTGGGTGGTTTCATAGCCACTGG-3′;
G385βR,
Forward (F):
SEQ ID NO: 15
5′-CGGGCCAACCcgcTCGCTGAACATC-3′;
Reverse (R):
SEQ ID NO: 16
5′-TCCTGGGTGGTTTCATAGCCACTGG-3′;

The primers were synthesized by a nucleic acid synthesis company, then dissolved in sterile water before proceeding with the protocol according to the kit instructions. As following:

### Step 1: Site-Directed Mutagenesis Via PCR

### PCR Reaction Mixture:

| Component | Volume | |
|---|---|---|
| Q5 Hot Start High-Fidelity 2X Master Mix | 12.5 | μL |
| 10 μM Forward Primer | 1.25 | μL |
| 10 μM Reverse Primer | 1.25 | μL |
| Template DNA (1-25 ng/μL) | 1 | μL |
| Nuclease-Free Water | 9.0 | μL |
| Total Volume | 25 | μL |

### Thermal Cycling Program:

| | | |
|---|---|---|
| Step | Temperature | Time |

| | | | | |
|---|---|---|---|---|
| Initial | 98° | C. | 30 | s |
| Denaturation | 98° | C. | 10 | s |
| 25 Cycles | 68° | C. | 10-30 | s |
| | 72° | C. | 62 | s |
| Final Extension | 72° | C. | 2 | min |

| | | | |
|---|---|---|---|
| Hold | 4-10° | C. | ∞ |

For mutants with ≥2 mutation sites, the PCR product from the prior mutation step was used as the template for subsequent rounds of site-directed mutagenesis.

### Step 2: Kinase, Ligase & DpnI (KLD) Treatment

Reaction mixture:

| Component | Volume | Final Concentration |
|---|---|---|
| PCR Product | 1 μL | |
| 2 × KLD Reaction Buffer | 5 μL | 1× |
| 10 × KLD Enzyme Mix | 1 μL | 1× |
| Nuclease-Free Water | 3 μL | |

Incubate at room temperature for 5 minutes.

### Step 3: Heat Shock Transformation

Add 5 μL of the KLD reaction mixture to 50 μL of chemically competent E. coli BL21(DE3) cells. Incubate on ice for 30 minutes, apply a 42° C. heat shock for 30 seconds, and return to ice for 5 minutes. Subsequently, add 950 μL of SOC sterile liquid medium and incubate at 37° C. with gentle shaking for 1 hour. Spread 40-100 μL of the bacterial suspension onto Kan50-LB agar plates and incubate overnight at 37° C. The resulting single colonies represent the mutant expression strains, designated as E. coli BL21(DE3)/pET28a-kcPA01˜18.

### Step 4: Mutant Verification

Inoculate the obtained mutant expression strains into 25 mL of LB liquid medium containing Kan50. Incubate at 37° C. overnight. Use a plasmid extraction kit to isolate the plasmid. Send the plasmid to a third-party biotech company for sequencing to confirm that the product is the intended point-mutated target product.

### 3. Expression of Wild-Type and Mutant KcPA

The recombinant strains E. coli BL21(DE3)/pET28a-kcPA and E. coli BL21(DE3)/pET28a-kcPA01˜18 were inoculated onto Kan50 LB agar plates and incubated in a 37° C. incubator for 12-16 hours. Single colonies were picked and inoculated into 25 mL of LB liquid medium containing Kan50. These cultures were grown overnight at 37° C. with shaking at 300 rpm. Next, 500 μL of the bacterial suspension was transferred to 50 mL of Kan50 LB liquid medium and incubated at 37° C. with shaking at 280 rpm. The OD600 was monitored, and when it reached 0.6-0.8, IPTG was added to a final concentration of 0.3 mM to induce protein expression. The cultures were induced for 10 hours at 25° C. with shaking at 220 rpm. The cells were harvested by centrifugation, and the cell pellets were resuspended in pre-chilled PBS buffer (pH 7.5) and kept on ice for 10 minutes. The resuspended cells were then centrifuged at 4° C., 12,000 rpm for 6 minutes to collect the cell pellets. The cell pellets were further resuspended in 50 mM PBS buffer (pH 7.5), and after another round of centrifugation at 4° C., 12,000 rpm for 6 minutes, the supernatant was discarded, and the final cell pellets were resuspended at a concentration of 0.01 g/mL. The cells were lysed using a sonicator under the following conditions: ice-water bath, 400 W power, with cycles of 3 seconds on and 5 seconds off for a total of 80 cycles. After lysis, the mixture was centrifuged at 4° C., 12,000 rpm for 15 minutes, and the supernatant was collected as the crude enzyme extract. SDS-PAGE was then used to analyze the expressed proteins.

FIG. 4 shows the SDS-PAGE image of expressed proteins. The lanes are labeled as follows: M: Protein marker. Lane 1: Supernatant of E. coli BL21(DE3)/pET28a. Lane 2: Supernatant of E. coli BL21(DE3)/pET28a-kcPA without induction. Lane 3: Supernatant of E. coli BL21(DE3)/pET28a-kcPA18 without induction. Lane 4: Supernatant of IPTG-induced E. coli BL21(DE3)/pET28a-kcPA18.

### Embodiment 2

### 1. Measurement of KcPGA Hydrolytic Activity

Principle of Measurement: Penicillin Potassium Salt (PGK) is hydrolyzed under the action of KcPA to produce 6-Aminopenicillanic Acid (6-APA) and phenylacetic acid. Under acidic conditions, 6-APA reacts with p-Dimethylaminobenzaldehyde (PDAB) to form a yellow-green substance that has a maximum absorption peak at 415 nm. The enzyme activity is defined as: in 0.1 M PBS buffer at 28° C., the amount of penicillin acylase required to catalyze the conversion of 20 mg/mL PGK into 1 mol of 6-APA per minute is defined as KcPA enzyme activity one unit (U).

Weigh 0.5 g of PGK and dissolve it in the aforementioned buffer solution, then adjust the volume to 25 mL. Pipette 2 mL of PGK solution into a centrifuge tube and add 0.1 mL of KcPA enzyme solution. Set up a control without adding KcPA while keeping all other conditions identical.

Place the reaction system in a water bath shaker at 28° C. and 200 rpm for 10 minutes. After the reaction, deactivate the enzyme by placing it in a 90° C. water bath for 2 minutes. Then, take 200 μL of the post-reaction solution and add it to 3 mL of 0.1 M citrate buffer at pH 3.0, followed by the addition of 1 mL of coloring reagent (0.5% PDAB). Allow it to stand at room temperature for 3 minutes before measuring the absorbance at 415 nm. Use the 6-APA standard curve to determine the concentration of 6-APA in the sample, and calculate the enzyme activity, i.e. the hydrolytic activity, using the formula provided.

Calculation Formula: Hydrolytic activity of penicillin acylase per mL:

$$U = \frac{{C}_{6 - APA} \times V}{t \times {V}_{E}}$$

Where, C_{6-APA}: Concentration of 6-APA in the sample, μmol/L; V: Volume of the reaction system, mL; V_{E}: Volume of penicillin acylase added, mL; t: Reaction time, 10 min.

### 2. Measurement of KcPA Synthesis Activity

6-Aminopenicillanic Acid (6-APA) and D-p-Hydroxyphenylglycine methyl ester (D-HPGM) are catalyzed by KcPA to synthesize Amoxicillin. The amount of Amoxicillin can be measured using High-Performance Liquid Chromatography (HPLC), thereby calculating the synthesis activity of Penicillin Acylase (PA). The enzyme activity is defined as: under certain conditions, the amount of enzyme required to catalyze the production of 1 μmol of Amoxicillin per minute is considered one unit of synthesis activity, denoted as U.

Weigh 1 g of 6-APA and 1.25 g of D-HPGM and dissolve them in 50 mL of 0.1 M PBS buffer at pH 6.3. Adjust the pH to 6.3 and then make up to a total volume of 100 mL with the same buffer. Add 0.1 mL of KcPA enzyme solution to this mixture and incubate at 25° C. with shaking at 200 rpm for 30 minutes. Inactivate the enzyme by placing it in a 90° C. water bath for 2 minutes. Filter 0.5 mL of the reaction mixture through a 0.22 μm aqueous filter membrane and dilute with phosphate buffer to 100 mL for HPLC analysis to determine the concentration of Amoxicillin. The formula for calculating enzyme activity is provided below:

Calculation Formula: Synthesis activity of penicillin acylase per mL:

$$U = \frac{{C}_{s a m p l e} \times V \times 200}{{V}_{E} \times t}$$

Where, Cₛₐₘₚₗₑ: Concentration of Amoxicillin, μmol/L; V: Volume of the reaction mixture, mL; 200: Dilution factor; V_{E}: Volume of enzyme added, mL; t: Reaction time, min.

HPLC Conditions: Agilent ZORBAX SB-C18 4.6×250 mm column, column temperature 25° C. Injection volume 10 μL. Mobile phase A (0.02 M NaH₂PO₄—Na₂HPO₄ buffer at pH 4.7), mobile phase B (methanol). Start with 90% mobile phase A and 10% mobile phase B for 5 minutes, increase mobile phase B from 10% to 50% between 5 and 7 minutes, maintain 50% for 10 minutes, reduce mobile phase B from 50% to 10% between 17 and 19 minutes, and finally equilibrate with 90% mobile phase A and 10% mobile phase B for 5 minutes. Total flow rate is 1 mL/min.

TABLE 2
Comparison of Activities Between Mutants and Wild Type

| | Comparison | Comparison |
|---|---|---|
| | of | of |
| | hydrolysis | Synthesis |
| Mutant ID | activity | Activity |
| KcPA | 100 | 100 |
| KcPA 01 | 580 | 1530 |
| KcPA 02 | 245 | 950 |
| KcPA 03 | 286 | 838 |
| KcPA 04 | 290 | 818 |
| KcPA 05 | 492 | 709 |
| KcPA 06 | 462 | 1118 |
| KcPA 07 | 745 | 2054 |
| KcPA 08 | 786 | 3037 |
| KcPA 09 | 664 | 2260 |
| KcPA | 100 | 100 |
| KcPA 10 | 858 | 3574 |
| KcPA 11 | 920 | 4380 |
| KcPA 12 | 730 | 3410 |
| KcPA 13 | 953 | 8842 |
| KcPA 14 | 1074 | 10568 |
| KcPA 15 | 620 | 4875 |
| KcPA 16 | 831 | 8640 |
| KcPA 17 | 1270 | 12680 |
| KcPA 18 | 1440 | 15620 |

Note:
The hydrolytic activity of recombinantly expressed wild-type KcPA is 15 U/mL (fermentation broth), and the synthesis activity is 80 U/mL. For ease of comparison, the enzyme activity of the wild-type KcPA is defined as 100 in Table 2, with each mutant's activity compared against this baseline.

From the table, it is evident that single-site mutants exhibit significantly enhanced hydrolytic and synthesis activities compared to the wild type, particularly F146αK on the α subunit and G385βR on the β subunit. Specifically, the single-point mutation F146αK shows 5.8 times higher hydrolytic activity and 15.3 times higher synthesis activity than the wild type. The G385βR mutant exhibits 4.6 times higher hydrolytic activity and approximately 11.2 times higher synthesis activity than the wild type. Comparing G385βY and G385βR mutants, the former has higher hydrolytic activity but not superior synthesis activity. When multiple mutations are combined, the enzyme activity increases significantly over single mutations, especially for the five-point mutant F146αK & F24βR & F71βY & N241βK & G385βR, which shows high levels of both hydrolytic and synthesis activities.

### Embodiment 3

### One-Step Synthesis of Amoxicillin Catalyzed by Wild-Type and Penicillin Acylase Mutants Using PGK

In PBS buffer at pH 7.0, PGK was added to reach a concentration of 200 mM, along with D-p-Hydroxyphenylglycine Methyl Ester (D-HPGM) to achieve a final concentration of 300 mM. Enzyme was added at 30 U/mL (based on synthetic activity). The reaction was carried out at 28° C. with constant stirring for 3 hours. After the reaction, HPLC analysis was performed to calculate the yield of Amoxicillin.

HPLC Conditions: Agilent ZORBAX SB-C18 4.6×250 mm column, column temperature 25° C. Injection volume 10 μL. Mobile phase A (0.02 M NaH₂PO₄—Na₂HPO₄ buffer at pH 4.7), mobile phase B (methanol). Start with 90% mobile phase A and 10% mobile phase B for 5 minutes, increase mobile phase B from 10% to 50% between 5 and 7 minutes, maintain 50% for 10 minutes, reduce mobile phase B from 50% to 10% between 17 and 19 minutes, and finally equilibrate with 90% mobile phase A and 10% mobile phase B for 5 minutes. Flow rate is 1 mL/min.

The reaction scheme is as follows:

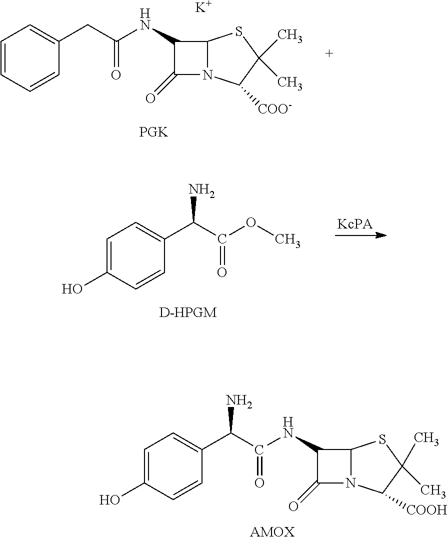

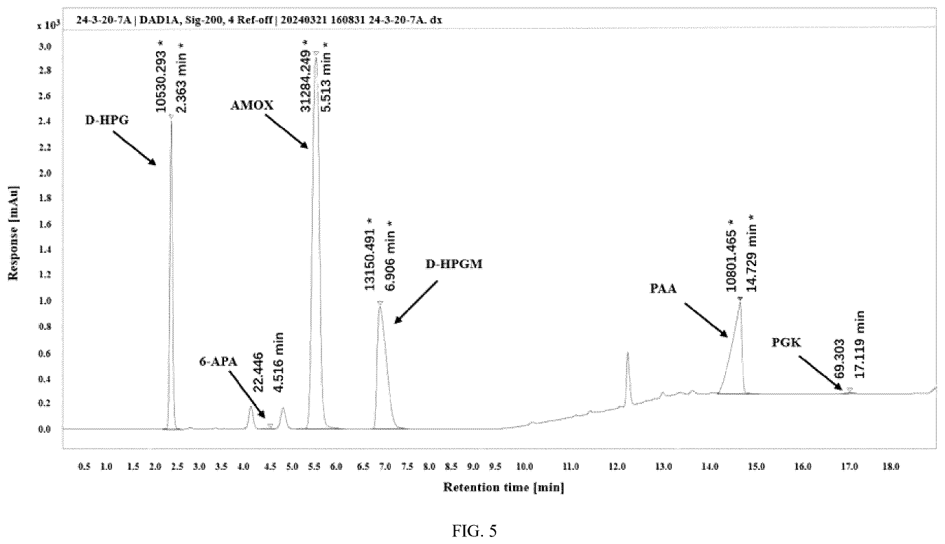

For mutant KcPA 18, the HPLC detection spectrum is shown in FIG. 5, where D-HPG represents D-p-Hydroxyphenylglycine, AMOX represents Amoxicillin, D-HPGM represents D-p-Hydroxyphenylglycine Methyl Ester, PAA represents Phenylacetic Acid, and PGK represents Penicillin Potassium Salt. As can be seen from the figure, the content of the intermediate 6-APA is extremely low, almost negligible.

TABLE 3
Yield of Amoxicillin Catalyzed by Various Mutants

| | Amoxicillin |
|---|---|
| Mutant ID | Yield/% |
| KcPA | 8.5 |
| KcPA 01 | 24.5 |
| KcPA 02 | 16.3 |
| KcPA 03 | 18.6 |
| KcPA 04 | 17.2 |
| KcPA 05 | 15.7 |
| KcPA 06 | 20.1 |
| KcPA 07 | 26.8 |
| KcPA 08 | 28.5 |
| KcPA 09 | 25.3 |
| KcPA | 8.5 |
| KcPA 10 | 29.2 |
| KcPA 11 | 34.7 |
| KcPA 12 | 28.9 |
| KcPA 13 | 68.2 |
| KcPA 14 | 85.6 |
| KcPA 15 | 37.7 |
| KcPA 16 | 67.9 |
| KcPA 17 | 92.8 |
| KcPA 18 | 99.0 |

From the results in the table above, it is evident that all mutants are capable of catalyzing the reaction between potassium salt of penicillin and D-Hydroxyphenylglycine Methyl Ester in one step to synthesize Amoxicillin within a single reaction system. Moreover, the product yields are significantly higher compared to the wild type.

### Embodiment 4

KcPA18-Catalyzed One-Step Synthesis of Amoxicillin from PGK

The reaction system was identical to that described in Example 3, with the sole exception that the PBS buffer in this example had a pH of 7.5. The HPLC analysis conditions were the same as those in Example 3.

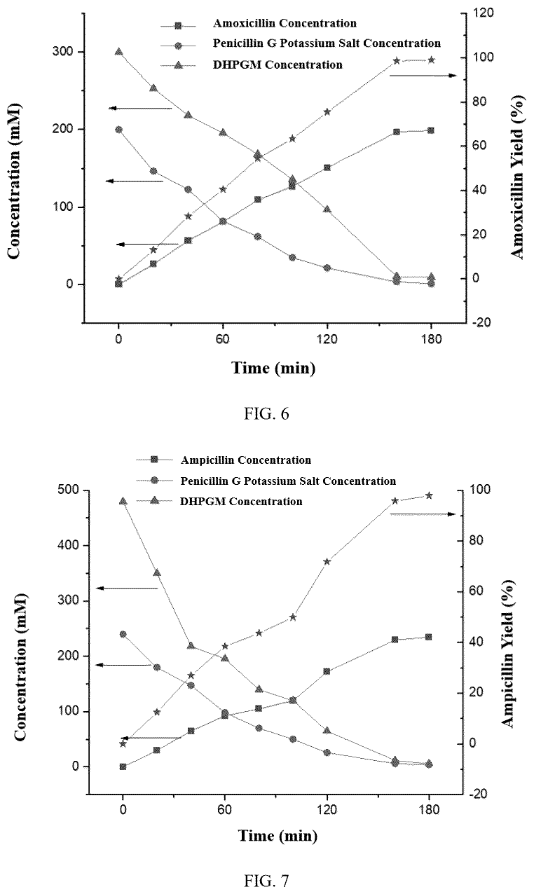

FIG. 6 shows the changes in the concentrations of the various substances during the reaction, which proceeded with a final yield of 98%.

### Embodiment 5

KcPA18-Catalyzed One-Step Synthesis of Ampicillin from PGK

To a PBS buffer (pH 7.5), PGK was added to a concentration of 240 mM, along with D-phenylglycine methyl ester (D-PGM) to a final concentration of 480 mM. The enzyme was added at a dosage of 30 U/mL (based on synthetic activity), and the reaction was carried out with constant temperature stirring at 25° C. for 3 hours. Samples were taken at regular intervals during the reaction for analysis.

The HPLC analysis conditions were the same as those in Example 3. The reaction equation is as follows:

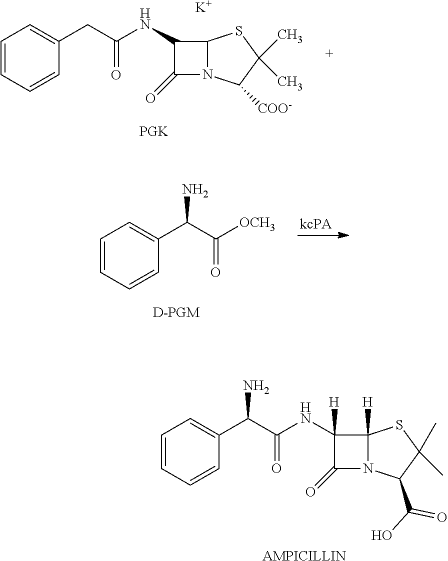

FIG. 7 shows the changes in the concentrations of the various substances during the reaction, which proceeded with a final yield of 98%.

### Embodiment 6

KcPA18-Catalyzed Synthesis of Cefaclor from 7-ACCA

To a PBS buffer (pH 7.5), 7-ACCA was added to a concentration of 200 mM, along with D-phenylglycine methyl ester (D-PGM) to a final concentration of 240 mM. The enzyme was added at a dosage of 20 U/mL (based on synthetic activity), and the reaction was carried out with constant temperature stirring at 15° C. for 2.25 hours. Samples were taken at regular intervals during the reaction for analysis.

The HPLC analysis conditions were as follows: a mobile phase of 0.01 M sodium phosphate (pH 6.8) and methanol (95:5, v/v) at a flow rate of 1.0 mL/min, using an Agilent ZORBAX SB-C18 column (4.6×250 mm), with an injection volume of 10 μL. The reaction equation is as follows:

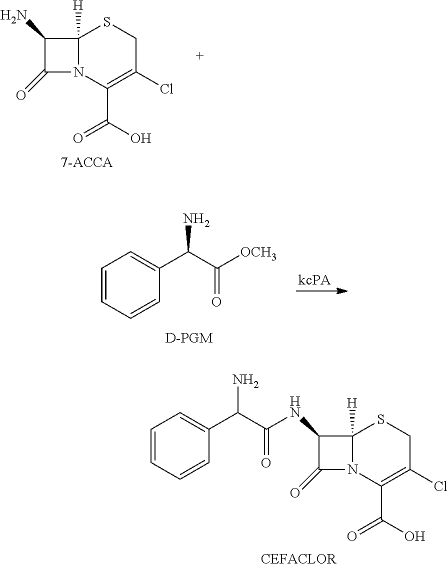 [SMILES: N[C@@H]1C(=O)N2C(C(=O)O)=C(Cl)CS[C@H]12 ; COC(=O)[C@H](N)c1ccccc1 ; NC(C(=O)N[C@@H]1C(=O)N2C(C(=O)O)=C(Cl)CS[C@H]12)c1ccccc1]

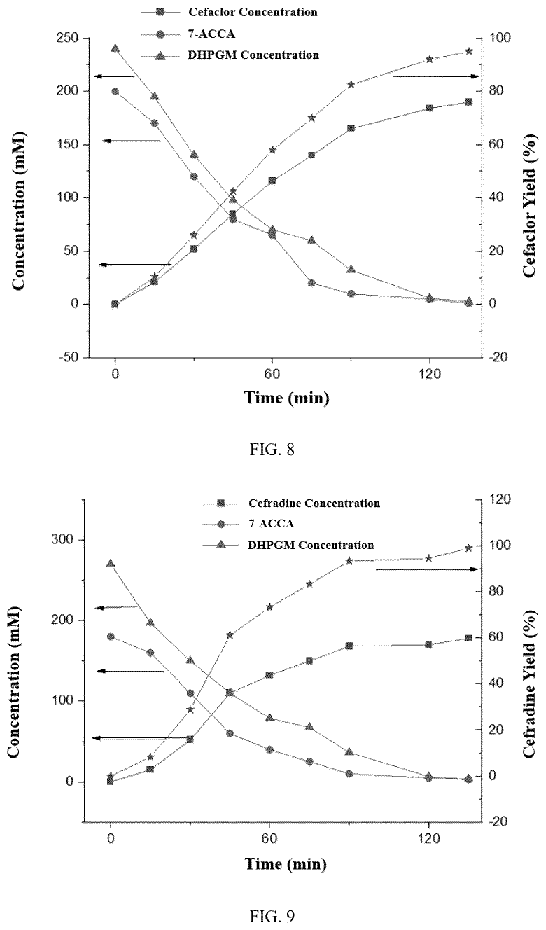

FIG. 8 shows the changes in the concentrations of the various substances during the reaction, which proceeded with a final yield of 95%.

### Embodiment 7

Synthesis of Cefradine from 7-ADCA Catalyzed by KcPA18

To a PBS buffer (pH 8.0), 7-ADCA was added to a concentration of 180 mM, along with D-phenylglycine methyl ester (D-PGM) to a final concentration of 270 mM. The enzyme was added at a dosage of 25 U/mL (based on synthetic activity), and the reaction was carried out with constant temperature stirring at 10° C. for 2.25 hours. Samples were taken at regular intervals for analysis.

The HPLC analysis conditions were as follows: mobile phase: 0.01 M sodium phosphate (pH 5.5) and methanol (93:7, v/v); flow rate: 1.0 mL/min; column: Agilent ZORBAX SB-C18 (4.6×250 mm); injection volume: 10 μL. The reaction equation is as follows:

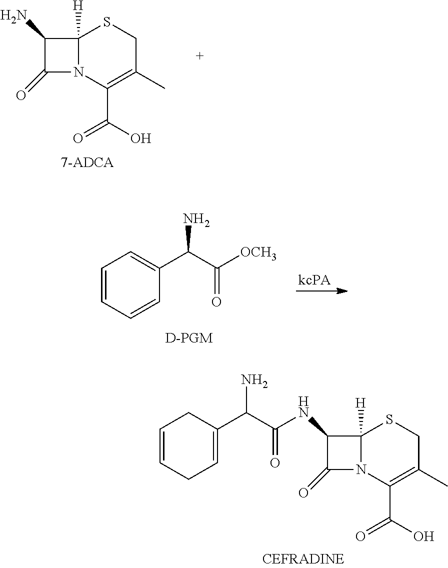 [SMILES: CC1=C(C(=O)O)N2C(=O)[C@@H](N)[C@H]2SC1 ; COC(=O)[C@H](N)c1ccccc1 ; CC1=C(C(=O)O)N2C(=O)[C@@H](NC(=O)C(N)C3=CCC=CC3)[C@H]2SC1]

FIG. 9 shows the changes in the concentrations of the various substances during the reaction, which proceeded with a final yield of 99%.

The above examples are merely preferred embodiments of the present invention and should not be construed as limiting the scope of the invention. Any modifications, substitutions, and improvements made within the spirit and principles of the invention are intended to be included within the scope of protection of the invention.

What is claimed is:

1. A mutant penicillin acylase, consisting of an amino acid sequence identical to SEQ ID NO: 1 except for a combination of mutations selected from the group consisting of: F146αK, F24βR, F71βY, N241βK, and G385βR.

2. A nucleotide encoding the mutant penicillin acylase according to claim 1.
